(19) United States
(12) Patent Application Publication
(10) Pub. No.: US 2013/0052724 A1
(43) Pub. Date: Feb. 28, 2013

(54) Ethanol and Protein Feed Plant Production System Using Whey Permeate

(71) Applicant: Joseph Van Groll, Marshfield, WI (US)

(72) Inventor: Joseph Van Groll, Marshfield, WI (US)

(21) Appl. No.: 13/222,755

(22) Filed: Aug. 31, 2011

(51) Int. Cl. C12M 1/00; C12M 1/02

(52) U.S. Cl. 435/289.1

(57) ABSTRACT

The present invention is a system for producing ethanol and yeast protein feed by fermenting whey permeate, a dairy waste product using four types of tanks: an inoculation tank, a seeding tank, a propagation tank, and a fermentation tank. Whey permeate and various chemicals are added to each of the tanks before being heated. The fermentation process for producing ethanol and yeast protein feed from whey permeate lasts 24 hours.

### FIELD OF INVENTION

The present invention relates to the field of ethanol production, and more specifically the production of ethanol and yeast protein feed from whey permeate.

### BRIEF DESCRIPTION OF THE DRAWINGS

FIG. 1 is an exemplary embodiment of an ethanol production system using whey permeate.

FIG. 2 is an exemplary embodiment of an inoculation tank.

FIG. 2a is an exemplary embodiment of an inoculation tank showi optional components.

FIG. 3 is an exemplary embodiment of a seeding tank.

FIG. 3a is an exemplary embodiment of a seeding tank showing optional components.

FIG. 4 is an exemplary embodiment of a propagation tank.

FIG. 4a is an exemplary embodiment of a propagation tank showing optional components

FIG. 5 in an exemplary embodiment of a fermentation tank.

FIG. 5a in an exemplary embodiment of a fermentation tank showing optional components.

### TERMS OF ART

As used herein, “adequate yeast cell count” means approximately 1 million cells per milliliter per tank contents.

As used herein, “agitator” refers to a tank agitation device for mixing and blending. An agitator may be used for wet and/or dry materials.

As used herein, the term “air compressor” refers to a device that converts power into kinetic energy by pressurizing and compressing air.

As used herein, the term “antibiotic” or “anti-microbial” means any anti-microbial agent developed for safe human and animal consumption including chlorine dioxide, hydrogen peroxide, phosphorus, hydrochloric acid, tetracycline, and synthetic antimicrobials effective against pathogenic bacteria resistant to current antimicrobials.

As used herein, the term “antimicrobial processes” refers to the best practices for inhibiting the growth of bacteria, which includes but is not limited to anti-microbial agents, temperature controlled structure, appropriately set timing devices, processes, and structures to avoid contamination (e.g., CIP practices).

As used herein, the term “Brix measurement device” means any component capable of measuring Brix levels, including but not limited to a hydrometer, a gas chromatograph (GC), and an HPLC. A Brix level may also be defined as a solids level near-infrared and combinations and equivalents thereof.

As used herein, the term “ethanol” refers to alcohol that is created by fermentation.

As used herein, the term “ethanol solution” refers to a solution comprised of any combination of ethanol and water.

As used herein, the term “fermentation” means the process whereby materials (e.g., lactose sugars) are broken down (e.g., by lactose-fermenting yeast) to create heat and alcohol.

As used herein, the term “hydrometer” refers to a Brix measuring instrument that measures the specific gravity (or relative density) of liquids; that is, the ratio of the density of the liquid to the density of water.

As used herein, the term “inoculation” and “inoculant” refers to processes and physical components directed to the introduction of a microorganism (e.g., a strain of yeast) onto a growth medium (e.g., whey permeate).

As used herein, the term “liquid” includes liquids, viscous solids, and liquids with suspended solids. A liquid may included mixtures into which solids are introduced.

As used herein, the term “propagation” refers to processes and physical components directed to exponential growth of yeast cultures on whey permeate.

As used herein, the term “seeding” refers to processes and physical components directed to the initiation of growth of yeast cultures on whey permeate.

As used herein, the term “separator” refers to a device that separates something into its constituent or distinct elements (e.g., yeast from ethanol water solution).

A “solid” is a non viscous material, and classifications of liquids and solids may overlap.

As used herein, the term “Venturi device” means any device known in the art that operates to reduce fluid pressure that results when air or fluid flows through a constricted section of pipe. A Venturi device mechanically incorporates air into the mixture by creating a pumping action by use of a pumping or vacuuming action. A Venturi device may or may not be operatively coupled to a pump and/or use gravity, may be located inside or outside of a tank, or may be used for liquids and solids. It combines the function of three pumps: recirculating, compressor, and transfer. The only pump needed is a circulating pump.

As used herein, the term “volumetric tank ratio” means the ratio of one tank size to the size of another tank, and may be any constant ratio so long as the yeast cell count is adequately maintained.

As used herein, the term “whey permeate” refers to a modified dairy product that is obtained by removing protein from whey.

As used herein, the term “yeast” means any micro-organism capable of converting carbohydrates to carbon dioxide and alcohols.

### BACKGROUND

Industrial processes for producing ethanol from whey permeate, a common waste byproduct of cheese production, are desirable because whey permeate is a pollutant, and ethanol can be used for biofuel. Land disposal of surplus whey can endanger the physical and chemical structures of soil, decrease crop yields, and cause serious water pollution. Disposal of surplus whey can be a significant expense because municipalities typically charge businesses for each gallon of their sewage. In addition, surcharges are often incurred for each pound of biochemical oxygen demand, a water pollutant contained in whey permeate. Other water pollutants in whey permeate include chemical oxygen demand, and some municipalities add surcharges for this waste as well. Creating products from whey reduces whey disposal costs for cheese producers, enabling their businesses to become more profitable. Thus, it is desirable to find inexpensive and environmentally-friendly alternatives for cheese producers to create commercially-desirable products from whey.

Current systems for the fermentation of whey typically require multiple batches, in some cases up to eight batches. Fermentation of each batch requires more than 24 hours. This means that the total fermentation process requires several days to produce biofuel.

Current methods also typically require multiple steps that increase production cost and time, e.g., monitoring and controlling pH of the whey permeate throughout the process.

It is desirable for a fermentation process to ferment ethanol from whey permeate in 24 hours using very few steps, because whey permeate can easily spoil or change to a temperature that is not useful for fermentation.

It is desirable for a system to produce ethanol from whey permeate using whey permeate unloaded from motor vehicles into holding tanks, because this permits fermentation at facilities located away from dairy plants that produce whey permeate as a waste byproduct.

### SUMMARY OF THE INVENTION

The present invention is a system for producing ethanol and yeast protein feed by fermenting whey permeate, a dairy waste product. Whey permeate is first pumped into the inoculation tank, and nutrient tubes add various chemicals and antimicrobials. The purpose of the inoculation tank is to introduce a strain of yeast that can ferment whey permeate. The contents of the inoculation tank are heated and transferred to a seeding tank, which initiates the growth of yeast cultures onto whey permeate. Whey permeate and the contents of the inoculation tank are pumped into the seeding tank, and various chemicals and antimicrobials are added by nutrient tubes. The contents of the seeding tank are heated before being transferred to the propagation tank, which exponentially grows yeast cultures on the whey permeate.

Whey permeate and the contents of the seeding tank are pumped into the propagation tank, and various chemicals and antimicrobials are added by nutrient tubes. The contents of the propagation tank are heated before being transferred to the fermentation tank. Whey permeate and the contents of the propagation tank are pumped into the fermentation tank, and various chemicals and antimicrobials are added by nutrient tubes. The contents of the fermentation tank are heated to ferment whey permeate into ethanol and a yeast protein feed.

The fermentation process for producing ethanol and yeast protein feed from whey permeate lasts 24 hours. Ethanol is useful as a biofuel, and the yeast protein feed can be used for ruminant animals. The system to produce ethanol from whey permeate can pump the ethanol from the fermentation tank to holding tanks for transport by motor vehicles.

### DETAILED DESCRIPTION OF INVENTION

For the purpose of promoting an understanding of the present invention, references are made in the text to exemplary embodiments of an industrial system producing ethanol from whey permeate, only some of which are described herein. It should be understood that no limitations on the scope of the invention are intended by describing these exemplary embodiments. One of ordinary skill in the art will readily appreciate that alternate but functionally equivalent processes for producing ethanol from whey permeate at an industrial scale may be used. The inclusion of additional elements may be deemed readily apparent and obvious to one of ordinary skill in the art. Specific elements disclosed herein are not to be interpreted as limiting, but rather as a basis for the claims and as a representative basis for teaching one of ordinary skill in the art to employ the present invention.

It should be understood that the drawings are not necessarily to scale; instead, emphasis has been placed upon illustrating the principles of the invention. In addition, in the embodiments depicted herein, reference numerals in the various drawings refer to identical or near identical structural elements.

Moreover, the terms “substantially” or “approximately” as used herein may be applied to modify any quantitative representation that could permissibly vary without resulting in a change in the basic function to which it is related. For example, one could make variations to the amounts of corn steep liquor and phosphorus nutrients used.

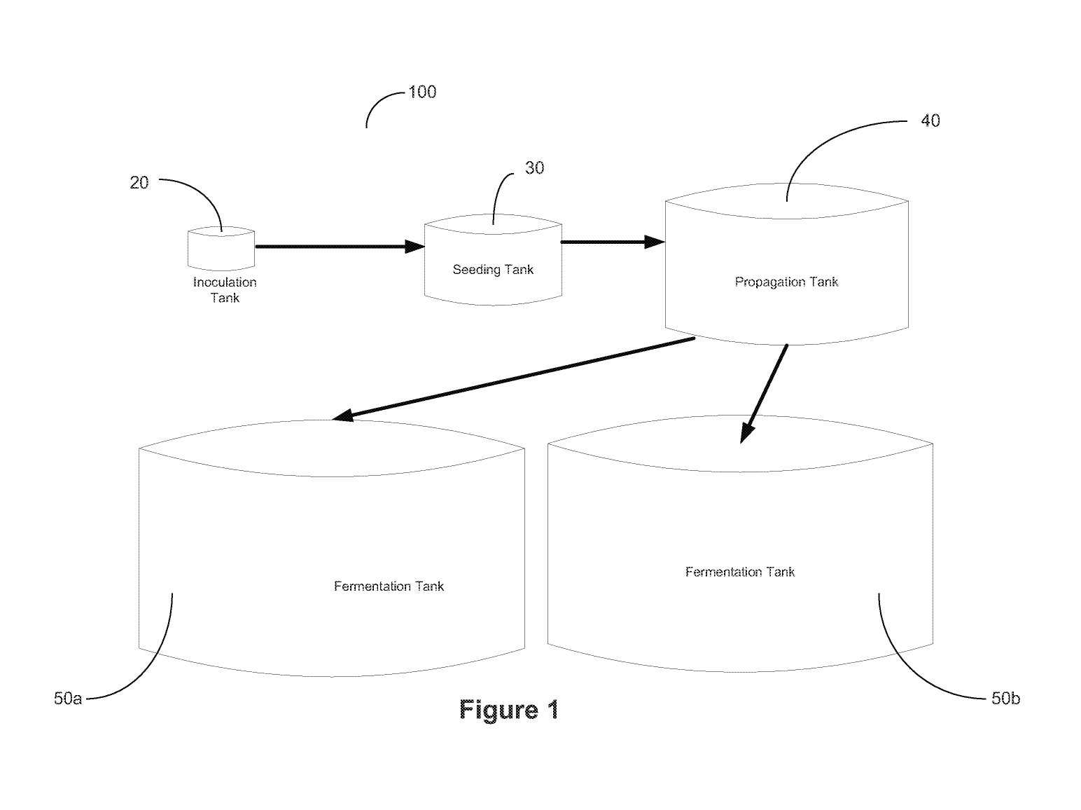

FIG. 1 is an exemplary embodiment of an ethanol production system that uses whey permeate 100. In the exemplary embodiment illustrated by FIG. 1, five tanks used to ferment whey permeate are shown; these include inoculation tank 20, seeding tank 30, propagation tank 40, and fermentation tanks 50a and 50b. The first tank used is inoculation tank 20, the purpose of which is to introduce a strain of yeast that can ferment whey permeate.

The ratio of the capacity of the tanks is a constant volumetric tank ratio. In the exemplary embodiment shown, inoculation tank 20 has an inner chamber possessing a capacity of 60 gallons. Seeding tank 30 has an inner chamber possessing a capacity of 600 gallons (or ten times the capacity of inoculation 20). Propagation tank 40 has an inner chamber possessing a capacity of 6,000 gallons (or ten times the capacity of inoculation 30). In the exemplary embodiment shown, fermentation tanks 50a and 50b each have an inner chamber possessing a capacity of 30,000 gallons, instead of a single 60,000 gallon fermentation tank, thus spotting the necessary volume (based on the propagation tank volume) into two tanks. When fermentation occurs in two tanks, the air flow (aeration) is optimized in this exemplary embodiment. Other embodiments may use more or fewer tanks.

Inoculation tank 20, seeding tank 30, and propagation tank 40 may be spot into smaller tanks so long as the volumetric tank ratio is preserved, and/or so long as the adequate yeast cell count is maintained.

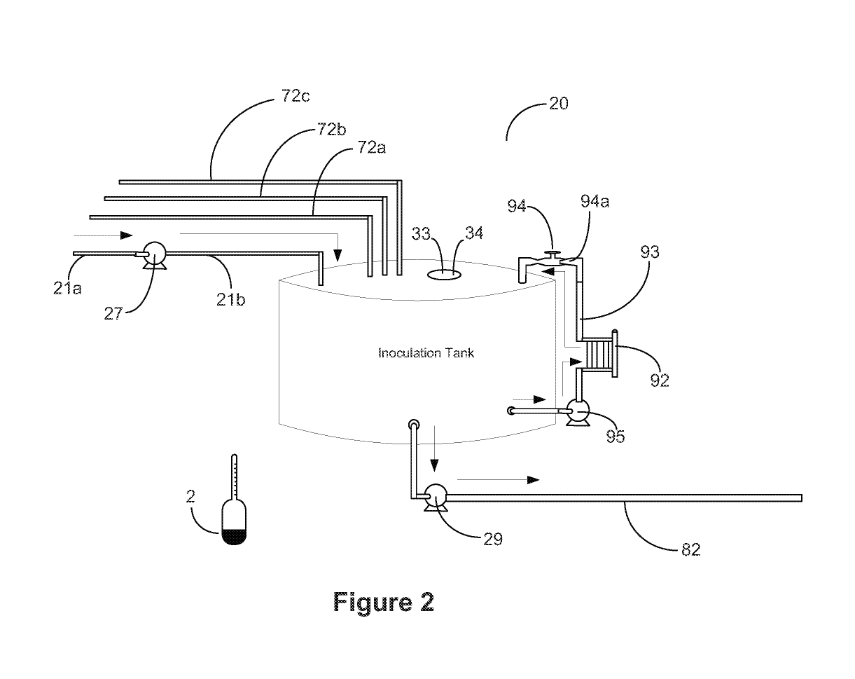

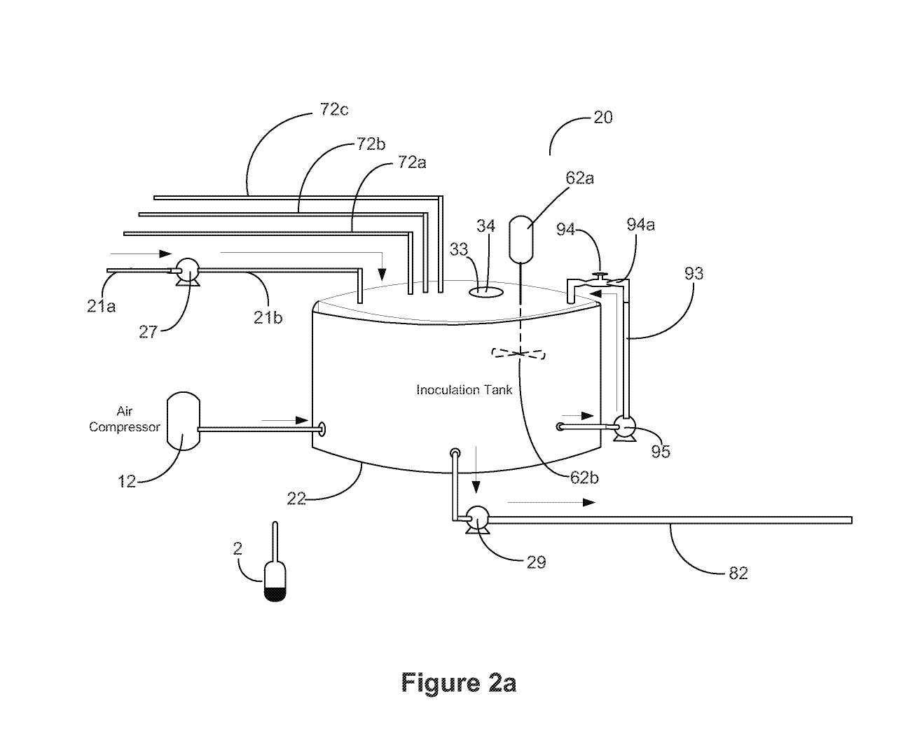

In the exemplary embodiment shown in FIG. 2, inoculation tank 20 is the first step tank into which materials are introduced during fermentation process. Inoculation tank 20 has an inner chamber with a capacity of 60 gallons. In further exemplary embodiments, inoculation tank 20 may have a capacity larger or smaller than 60 gallons. In further exemplary embodiments, additional inoculation tanks may be used.

As shown in FIG. 2, pipe 21a unloads whey permeate from a truck in deposits of 55 gallons, 11 to 12 percent of which is solid, into inoculation tank 20. Pipe 21b conducts the whey permeate from unloading pump 27 to inoculation tank 20. In the embodiment shown, unloading pump 27 is a centrifugal pump known in the art. In further exemplary embodiments, deposits of more or fewer gallons may be used.

As shown in FIG. 2, Venturi pipe 94 is a Venturi device with constricted section 94a that produces a Venturi effect. Constricted section 94a has a circumference of three quarters of an inch, and the tubular body of Venturi pipe 94 has a circumference of 2-4 inches. Tubular diameter dimensions will increase as tank size increases. The embodiment shown includes vertical air feed tube 93 that transfers air into inoculation tank 20.

A Venturi device mechanically incorporates air into the mixture by creating a pumping action by use of a pumping or vacuuming action. In the exemplary embodiment shown, Venturi pipe 94 may or may not be operatively coupled to a Venturi pump 95 and/or use gravity to induce flow. A Venturi device may be located inside or outside of a tank and may be used for liquids and solids.

FIG. 2 further illustrates nutrient tubes 72a, 72b, and 72c to convey nutrients into inoculation tank 20. Nutrient tube 72a conveys nitrogen (e.g., urea ammonium nitrate), nutrient tube 72b conveys phosphorus (e.g., liquid ammonium phosphate), and nutrient tube 72c conveys corn steep liquor. In the embodiment shown, these nutrients are commercially available.

The contents of inoculation tank 20 are then brought to the approximate temperature of 85 degrees by heat exchanger assembly 92, which is commercially available and known in the art. In the embodiment shown, heat exchanger assembly 92 includes a temperature measuring component, heating components, and a cleaning component. Once the proper temperature has been reached, a quantity of a strain of yeast (e.g., Kluyveromyces marxianus) is manually introduced into inoculation tank 20 at a ratio of 1 gallon per 60 gallons. Further exemplary embodiments may use different quantities of Kluyveromyces marxianus or different strains of yeast culture.

Antimicrobial port 33 is a port for introducing antimicrobials into inoculation tank 20. Port 34 is for adding an actual inoculant. In various embodiments, both may be added through the same or different ports, and in still other embodiments, ports 33 and 34 could be a tube or other receptacle. Further embodiments need not require anti-microbial agents if microbial levels are minimal.

The contents of inoculation tank 20 are then checked for the liquid to solid ratio and Brix level with sugar hydrometer 2, which is a Brix measurement device. In this exemplary embodiment, this first occurs after eight hours and at every four hours thereafter. At the correct time, the contents of inoculation tank 20 are transferred to seeding tank 30 by transfer pump 29 through discharge tube outlet 82.

FIG. 2a illustrates an alternative exemplary embodiment of inoculation tank 20 that demonstrates optional components that can be added to inoculation tank 20 illustrated in FIG. 2 if needed.

Air compressor 12 supplies additional air if the air provided by Venturi device 94 is inadequate. In further embodiments, a blower may be used instead of air compressor 12.

Agitator 62b with agitator motor 62a stirs the contents of inoculation tank 20.

Thermal tank jacket 22 can be added around inoculation tank 20 in place of heat exchanger 92 (not shown).

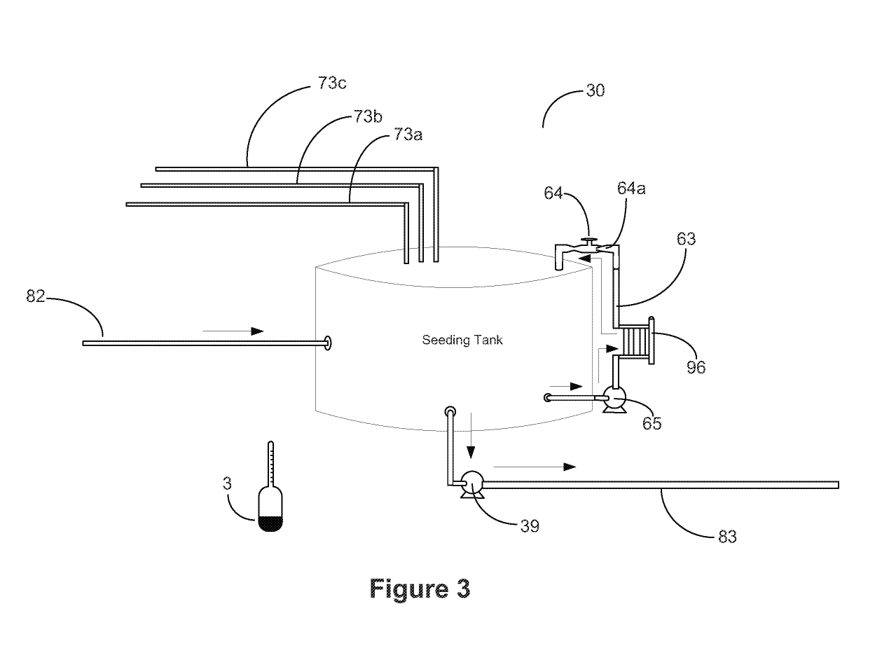

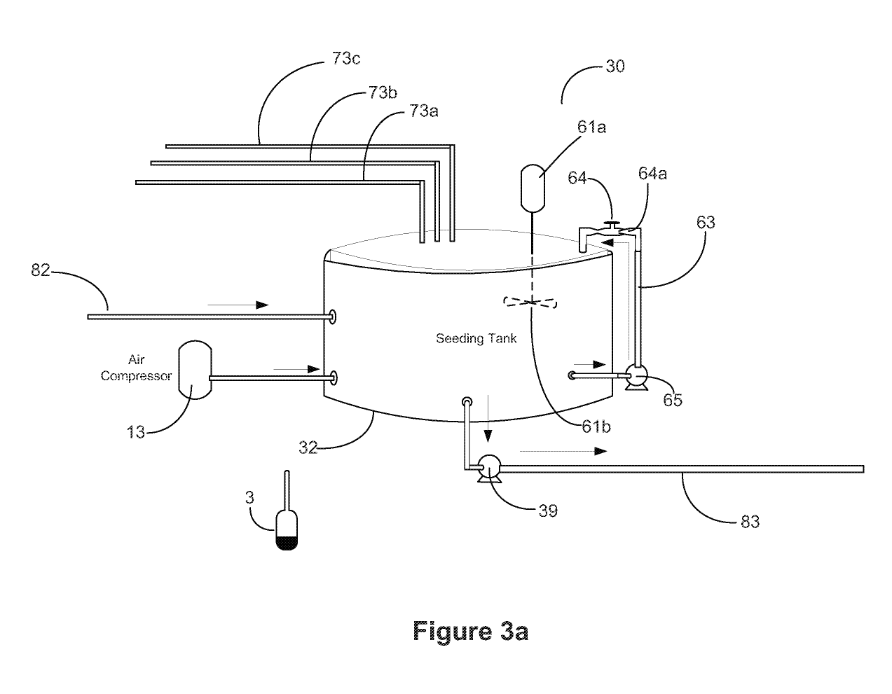

In the exemplary embodiment shown in FIG. 3, seeding tank 30 is the second step tank into which materials are introduced during the fermentation process. Seeding tank 30 has an inner chamber with a capacity of 600 gallons. In further exemplary embodiments, seeding tank 30 may have a capacity larger or smaller than 600 gallons. In further exemplary embodiments, additional seeding tanks may be used. As shown in FIG. 3, seeding tank 30 receives the contents of inoculation tank 20 (not shown) through discharge tube outlet 82.

As shown in FIG. 3, Venturi pipe 64 is a Venturi device with constricted section 64a that produces a Venturi effect. Tubular diameter dimensions of Venturi pipe 64 and constricted section 64a will vary depending on the size of seeding tank 30. The embodiment shown includes vertical air feed tube 63 that transfers air into seeding tank 30.

In the exemplary embodiment shown, Venturi pipe 64 may or may not be operatively coupled to a Venturi pump 65 and/or use gravity to induce flow. A Venturi device may be located inside or outside of a tank and may be used for liquids and solids.

FIG. 3 further illustrates nutrient tubes 73a, 73b, and 73c to convey nutrients into seeding tank 30. Nutrient tube 73a conveys nitrogen (e.g., urea ammonium nitrate), nutrient tube 73b conveys phosphorus (e.g., liquid ammonium phosphate), and nutrient tube 73c conveys corn steep liquor. In the embodiment shown, these nutrients are commercially available.

In the exemplary embodiment shown in FIG. 3, one ounce of antimicrobial feed and one quarter gallon of hydrogen peroxide are fed into seeding tank 30. Further exemplary embodiments may use different amounts of antimicrobial feed or hydrogen peroxide, depending on the amount of whey permeate that is used. Still further embodiments may use a different antimicrobial feed. In other exemplary embodiments, different amounts of antimicrobials and hydrogen peroxide may be used depending on the amount of the other nutrients used. Further embodiments need not require anti-microbial agents if microbial levels are minimal.

The contents of seeding tank 30 are then brought to the approximate temperature of 88 degrees by heat exchanger assembly 96, which is commercially available and known in the art. In the embodiment shown, heat exchanger assembly 96 includes a temperature measuring component, heating components, and a cleaning component.

The contents of seeding tank 30 are then checked for the liquid to solid ratio and Brix level with sugar hydrometer 3, which is a Brix measurement device. At the correct time, the contents of seeding tank 30 are transferred to propagation tank 40 by transfer pump 39 through discharge tube outlet 83.

FIG. 3a illustrates an alternative exemplary embodiment of seeding tank 30 that demonstrates optional components that can be added to seeding tank 30 illustrated in FIG. 3 if needed.

Air compressor 13 supplies additional air if the air provided by Venturi device 64 is inadequate. In further embodiments, a blower may be used instead of air compressor 13.

Agitator 61b with agitator motor 61a stirs the contents of seeding tank 30.

Thermal tank jacket 32 can be added around seeding tank 30 replacing heat exchanger 96 (not shown).

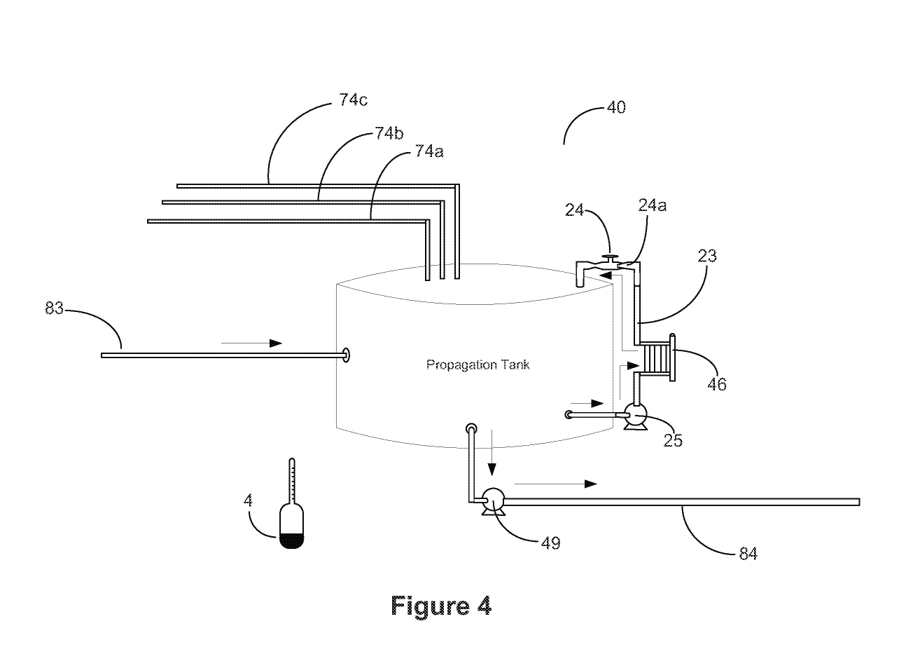

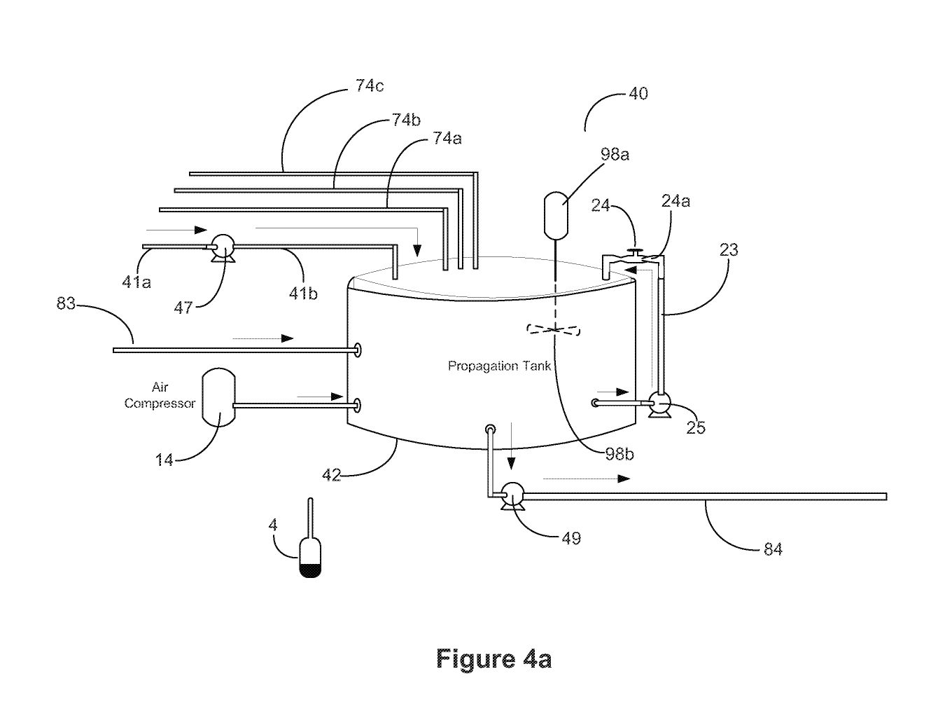

In the exemplary embodiment shown in FIG. 4, propagation tank 40 is the third step tank into which materials are introduced during the fermentation process. Propagation tank 40 has an inner chamber with a capacity of 6,000 gallons. In further exemplary embodiments, propagation tank 40 may have a capacity larger or smaller than 6,000 gallons. In further exemplary embodiments, additional propagation tanks may be used.

Propagation tank 40 receives the contents of seeding tank 30 (not shown) through discharge tube outlet 83.

As shown in FIG. 4, Venturi pipe 24 is a Venturi device with constricted section 24a that produces a Venturi effect. Tubular diameter dimensions of Venturi pipe 24 and constricted section 24a will vary depending on the size of propagation tank 40. The embodiment shown includes vertical air feed tube 23 that transfers air into propagation tank 40.

In the exemplary embodiment shown, Venturi pipe 24 may or may not be operatively coupled to a Venturi pump 25 and/or use gravity to induce flow. A Venturi device may be located inside or outside of a tank and may be used for liquids and solids.

FIG. 4 further illustrates nutrient tubes 74a, 74b, and 74c to convey nutrients into propagation tank 40. Nutrient tube 74a conveys nitrogen (e.g., urea ammonium nitrate), nutrient tube 74b conveys phosphorus (e.g., liquid ammonium phosphate), and nutrient tube 74c conveys corn steep liquor. In the embodiment shown, these nutrients are commercially available.

In the exemplary embodiment shown in FIG. 4, two ounces of antimicrobial feed and one quarter gallon of hydrogen peroxide are fed into propagation tank 40. Further exemplary embodiments may use different amounts of antimicrobial feed or hydrogen peroxide, depending on the amount of whey permeate that is used. Still further embodiments may use a different antimicrobial feed. In other exemplary embodiments, different amounts of antimicrobials and hydrogen peroxide may be used depending on the amount of the other nutrients used. Further embodiments need not require anti-microbial agents if microbial levels are minimal.

The contents of propagation tank 40 are then brought to the approximate temperature of 90-91 degrees by heat exchanger assembly 46, which is commercially available and known in the art. In the embodiment shown, heat exchanger assembly 46 includes a temperature measuring component, heating components, and a cleaning component.

The contents of propagation tank 40 are then checked for the liquid to solid ratio and Brix level with sugar hydrometer 4, which is a Brix measurement device. At the correct time, the contents of propagation tank 40 are transferred to fermentation tank 50 by transfer pump 49 through discharge tube outlet 84.

FIG. 4a illustrates an alternative exemplary embodiment of propagation tank 40 that demonstrates optional components that can be added to the inoculation tank 40 illustrated in FIG. 4 if needed.

Air compressor 14 supplies additional air if the air provided by Venturi device 24 is inadequate. In further embodiments, a blower may be used instead of air compressor 14.

Agitator 98b with agitator motor 98a stirs the contents of propagation tank 40.

Thermal tank jacket 42 can be added around propagation rank 40 replacing heat exchanger 46 (not shown).

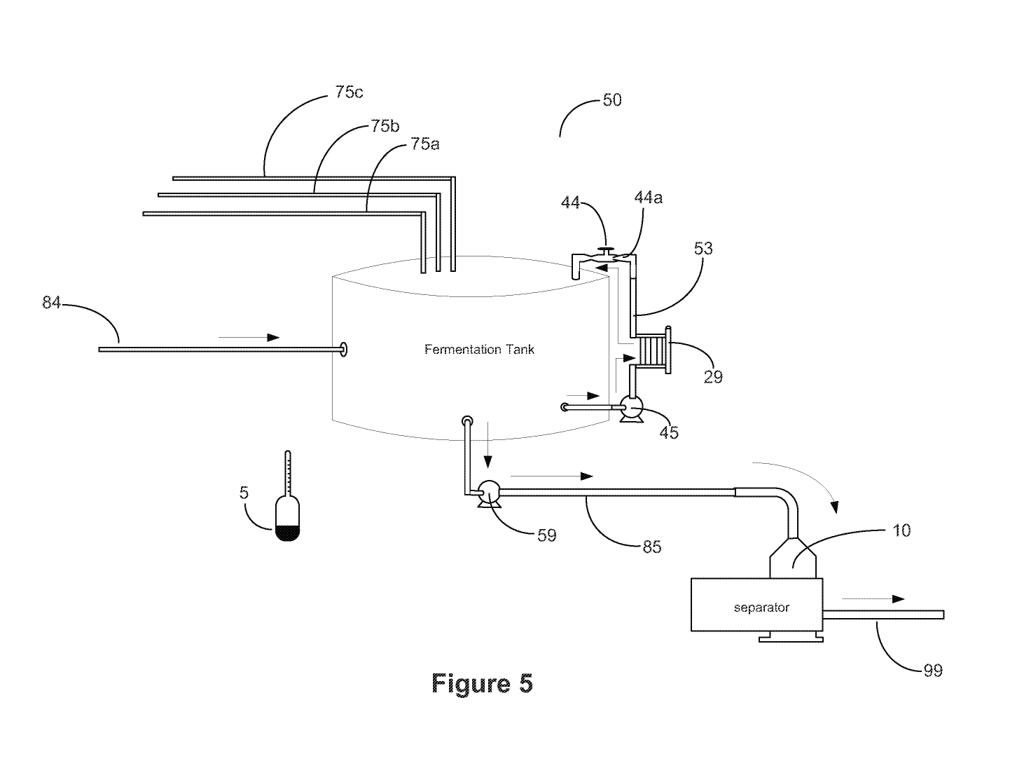

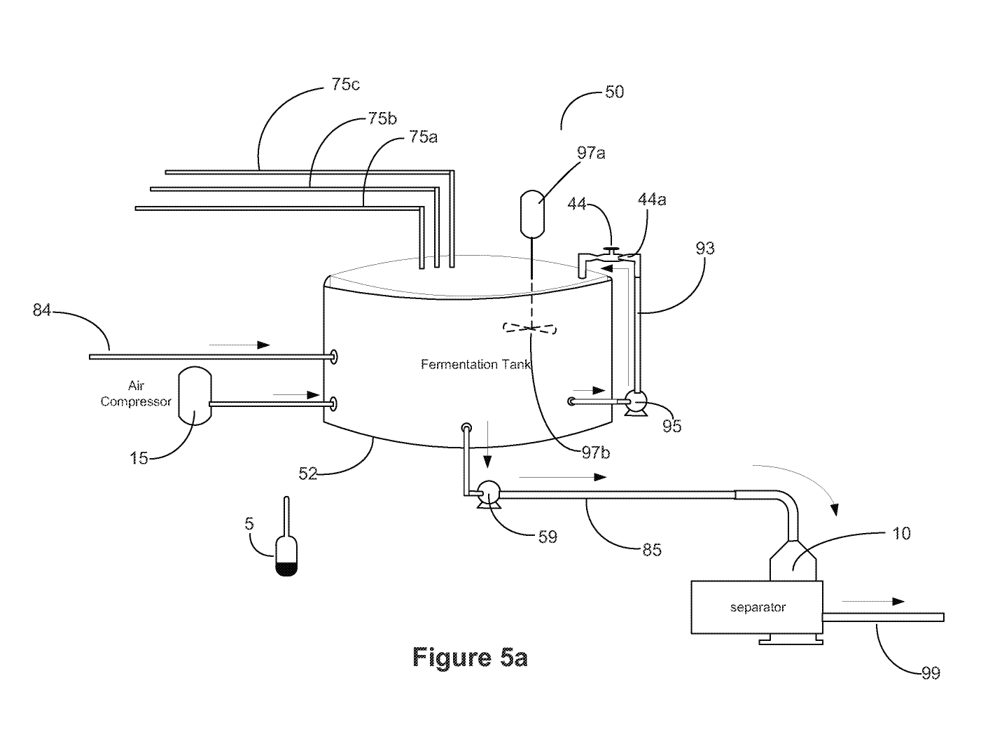

In the exemplary embodiment shown in FIG. 5, fermentation tank 50 is the fourth and final step tank into which materials are introduced during the fermentation process. Fermentation tank 50 has an inner chamber with a capacity of 30,000 gallons. In further exemplary embodiments, fermentation tank 50 may have a capacity larger or smaller than 30,000 gallons, depending on the desired production level for ethanol and yeast protein feed. In further exemplary embodiments, additional fermentation tanks may be used. As shown in FIG. 5, Fermentation tank 50 receives the contents of propagation tank 40 (not shown) through discharge tube outlet 84.

As shown in FIG. 5, Venturi pipe 44 is a pipe with constricted section 44a that produces a Venturi effect. Tubular diameter dimensions of Venturi pipe 44 and constricted section 44a will vary depending on the size of fermentation tank 50. The embodiment shown includes vertical air feed tube 53 that transfer air into fermentation tank 50.

In the exemplary embodiment shown, Venturi pipe 44 may or may not be operatively coupled to a Venturi pump 45 and/or use gravity to induce flow. A Venturi device may be located inside or outside of a tank and may be used for liquids and solids.

FIG. 5 further illustrates nutrient tubes 75a, 75b, and 75c that convey nutrients into fermentation tank 50. Nutrient tube 75a conveys nitrogen (e.g., urea ammonium nitrate), nutrient tube 75b conveys phosphorus (e.g., liquid ammonium phosphate), and nutrient tube 75c conveys corn steep liquor. In the embodiment shown, these nutrients are commercially available.

In the embodiment shown in FIG. 5, five ounces of antimicrobial feed are fed into fermentation tank 50. Further exemplary embodiments may use different amounts of antimicrobial feed or hydrogen peroxide, depending on the amount of whey permeate that is used. Still further embodiments may use a different antimicrobial feed. In other exemplary embodiments, different amounts of antimicrobials and hydrogen peroxide may be used depending on the amount of other nutrients used. Further embodiments need not require anti-microbial agents if microbial levels are minimal.

The contents of fermentation tank 50 are then brought to the approximate temperature of 95-98 degrees by heat exchanger assembly 29, which is commercially available and known in the art. In the embodiment shown, heat exchanger assembly 29 includes a temperature measuring component, heating components, and a cleaning component.

The contents of fermentation tank 50 are then checked for the liquid to solid ratio and Brix level with sugar hydrometer 5, which is a Brix measurement device. At the correct time, the contents of fermentation tank 50 are transferred to separator 10 to by transfer pump 59 through discharge tube outlet 85. In separator 10, ethynol is separated from yeast protein. The ethanol is then pumped through collection pipe 99 into holding tanks (not shown).

FIG. 5a illustrates an alternative exemplary embodiment of fermentation tank 50 that demonstrates optional components that can be added to fermentation tank 50 illustrated in FIG. 5 if needed.

Air compressor 15 supplies additional air if the air provided by Venturi device 44 is inadequate. In further embodiments, a blower may be used instead of air compressor 15.

Agitator 97b with agitator motor 97a stirs the contents of fermentation tank 50.

Thermal tank jacket 52 can be added around fermentation tank 50 replacing heat exchanger 29 (not shown).

What is claimed is:

1. An ethanol production apparatus comprised of:
at least one separator;
at least one ethanol collection pipe;
at least one inoculation tank having an inner chamber;
at least one seeding tank;
at least one propagation tank; and
at least one fermentation tank,
wherein said at least one inoculation tank, at least one seeding tank, at least one propagation tank, and at least one fermentation tank are in a constant volumetric ratio to each other.

2. The apparatus of claim 1 wherein said at least one seeding tank has a ratio of ten times the size of said at least one inoculation tank.

3. The apparatus of claim 1 wherein said at least one propagation tank has a ratio of ten times the size of said at least one seeding tank.

4. The apparatus of claim 1 wherein said at least one fermentation tank has a ratio of at least five times the size of said at least one propagation tank.

5. The apparatus of claim 1 wherein said at least one inoculation tank is comprised of:
at least one inner chamber having a capacity of at least 60 gallons, wherein the internal temperature of said internal chamber has a temperature range of 85 to 95 degrees Fahrenheit;
at least one Brix measurement device which indicates when the Brix level is between 2.5 and 3.5;
at least one nutrient tube for depositing at least one nitrogen nutrient;
at least one port for depositing at least one strain of yeast;
at least one port for depositing antimicrobial agents; and
at least one Venturi device.

6. The apparatus of claim 5 which further includes:
at least one nutrient tube for depositing at least one phosphorus nutrient; and
at least nutrient tube for depositing corn steep liquor.

7. The apparatus of claim 5 which further includes at least one agitator.

8. The apparatus of claim 5 which further includes at least one component selected from a group consisting of a heat exchanger or an insulating tank jacket.

9. The apparatus of claim 5 which further includes at least one air compressor to supply air pressure.

10. The apparatus of claim 5 wherein said at least one antimicrobial agent is selected from the group consisting of antimicrobial alternatives approved for safe human and animal consumption, chlorine dioxide, hydrogen peroxide, phosphorus, hydrochloric acid, tetracycline, and synthetic antimicrobials effective against pathogenic bacteria resistant to current antimicrobials.

11. The apparatus of claim 1 wherein said at least one seeding tank is comprised of:
at least one inner chamber having a capacity ratio of at least ten times the volume of said at least one inoculation tank and a temperature range of 85 to 95 degrees Fahrenheit;
at least one Brix measurement device which indicates when the Brix level is between 2.5 and 3.5;
at least one nutrient tube for depositing at least one nitrogen nutrient;
at least one port for depositing antimicrobial agents; and
at least one Venturi device.

12. The apparatus of claim 11 which further includes:
at least one nutrient tube for depositing at least one phosphorus nutrient; and
at least nutrient tube for depositing corn steep liquor.

13. The apparatus of claim 11 which further includes at least one agitator.

14. The apparatus of claim 11 which further includes at least one component selected from a group consisting of a heat exchanger or an insulating tank jacket.

15. The apparatus of claim 11 which further includes at least one air compressor to supply air pressure.

16. The apparatus of claim 11 wherein said at least one antimicrobial agent is selected from the group consisting of penicillin alternatives approved for safe human and animal consumption, chlorine dioxide, hydrogen peroxide, phosphorus, hydrochloric acid, tetracycline, and synthetic antimicrobials effective against pathogenic bacteria resistant to current antimicrobials.

17. The apparatus of claim 1 wherein said at least one propagation tank is comprised of:
at least one inner chamber having a capacity ratio of at least ten times the volume of said at least one seeding tank and a temperature range of 85 to 95 degrees Fahrenheit;
at least one Brix measurement device which indicates when the Brix level is between 2.5 and 3.5;
at least one nutrient tube for depositing at least one nitrogen nutrient;
at least one port for depositing antimicrobial agents; and
at least one Venturi device.

18. The apparatus of claim 17 which further includes:
at least one nutrient tube for depositing at least one phosphorus nutrient; and
at least nutrient tube for depositing corn steep liquor.

19. The apparatus of claim 17 which further includes at least one agitator.

20. The apparatus of claim 17 which further includes at least one component selected from a group consisting of a heat exchanger or an insulating tank jacket.

21. The apparatus of claim 17 which further includes at least one air compressor to supply air pressure.

22. The apparatus of claim 17 wherein said at least one antimicrobial agent is selected from the group consisting of penicillin alternatives approved for safe human and animal consumption, chlorine dioxide, hydrogen peroxide, phosphorus, hydrochloric acid, tetracycline, and synthetic antimicrobials effective against pathogenic bacteria resistant to current antimicrobials.

23. The apparatus of claim 1 wherein said at least one fermentation tank is comprised of:
at least one inner chamber having a capacity ratio of at least five times the volume of said at least one propagation tank and a temperature range of 95 to 98 degrees Fahrenheit;
at least one Brix measurement device which indicates when the Brix level is between 2.5 and 3.5;
at least one nutrient tube for depositing at least one nitrogen nutrient;
at least one port for depositing antimicrobial agents; and
at least one Venturi device.

24. The apparatus of claim 23 which further includes:
at least one nutrient tube for depositing at least one phosphorus nutrient; and
at least one nutrient tube for depositing corn steep liquor.

25. The apparatus of claim 23 which further includes at least one agitator.

26. The apparatus of claim 23 which further includes at least one component selected from a group consisting of a heat exchanger or an insulating tank jacket.

27. The apparatus of claim 23 which further includes at least one air compressor to supply air pressure.

28. The apparatus of claim 23 wherein said at least one antimicrobial agent is selected from the group consisting of penicillin alternatives approved for safe human and animal consumption, chlorine dioxide, hydrogen peroxide, phosphorus, hydrochloric acid, tetracycline, and synthetic antimicrobials effective against pathogenic bacteria resistant to current antimicrobials.
